(19) United States
(12) Patent Application Publication
(10) Pub. No.: US 2008/0027425 A1
(43) Pub. Date: Jan. 31, 2008

(54) Varicosis Treatment Apparatus

(76) Inventors: Kazumasa Orihashi, Hiroshima (JP); Shuro Hayashi, Hiroshima (JP)

Correspondence Address: Nixon Peabody, 401 9th Street, N.W., Suite 900, Washington, DC, 20004-2128, US

(21) Appl. No.: 11/661,124

(22) PCT Filed: Aug. 30, 2005
(86) PCT No.: PCT/JP05/15733

§ 371 Date: Feb. 26, 2007

(30) Foreign Application Priority Data
Sep. 1, 2004 (JP) 2004-253957

(51) Int. Cl. A61B 18/14

(52) U.S. Cl. 606/41

(57) ABSTRACT

A puncturing member 2 including a puncturing part 2a for puncturing a vein in which a varicosis is formed is formed of a conductor. A counter electrode plate 5 to be allowed to be in contact with the surface of an organism and a foot switch 18 are connected to an apparatus main body 4. An inverter circuit for generating high frequency power is provided in the apparatus main body 4. After the end part of a cylinder member 3 is allowed to puncture skin, the puncturing member 2 is inserted into the cylinder member 3 so that the puncturing part 2a punctures the vein in which the varicosis is formed. The high frequency power generated at the inverter circuit is supplied to the puncturing part 2a. Heat generated at the puncturing part 2a cauterizes the vein to block the bloodstream, thereby degenerating the vein.

### TECHNICAL FIELD

The present invention relates to a treatment apparatus for treating a varicosis formed in a vein of an organism.

### BACKGROUND ART

Multiple veins are distributed in dermis and hypodermal tissue which compose skin of an organism. Injury, for example, valve failure or the like may occur in a vein distributed in the skin to form a varicosis in some cases. When a varicosis is formed in a vein in the skin, the vein is liable to be raised in the surface of the skin in a webbed or reticular state to be conspicuous, which necessitates a varicosis treatment especially in cosmetic view. As one of treatment methods, a sclerotherapy disclosed in, for example, Patent Document 1 has been known conventionally. The sclerotherapy is a method in which a sclerosing agent composed of a medicine that causes injury in organic tissue is injected into a vein in which a varicosis is formed with the use of an injection syringe to cause injury in the inner wall of the vein and the components of the blood, thereby forming a thrombus. The thus formed thrombus blocks the bloodstream in the vein to harden and degenerate the vein in which the bloodstream is blocked, so that the raised vein fades away from the surface of the skin.
Patent Document 1: Japanese Patent Application Laid Open Publication No. 8-98858A (Page 2)

### SUMMARY OF THE INVENTION

Problems that the Invention is to Solve

The sclerosing agent used for the sclerotherapy in Patent Document 1, however, causes injury in the organic tissue, and therefore, a defense reaction of the organism is exerted excessively to cause inflammation in the vein or the organic tissue therearound. Further, the sclerosing agent after injection into the vein diffuses through the bloodstream in the vein to a part that requires no treatment, widening the inflamed part undesirably. When the inflammation is caused over a wide range in the vicinity of the surface of the skin, skin injury, such as storage disease where a pigment is deposited in the skin or the like is liable to be caused, which would result in cosmetically poor effects of the treatment. As well, because a plenty of sympathetic nerves are distributed in the skin, wide-ranged inflammation would cause ache.

The present invention has been made in view of the foregoing and has its object of performing treatment for a varicosis formed in a vein distributed in skin of which effects are cosmetically excellent and which causes less or no pain by blocking the bloodstream in the vein without using a sclerosing agent.

Means for Solving the Problems

To attain the above object, in the present invention, a puncturing member is allowed to puncture a vein in which a varicosis is formed or organic tissue in the vicinity thereof, and electric power is supplied thereto to coagulate the vein electrically for occluding the vein.

Specifically, in a first invention, a varicosis treatment apparatus includes: a puncturing member including a puncturing part for puncturing a vein in which a varicosis is formed or an organic tissue in the vicinity of the vein, at least a part of the puncturing part being formed of a conductor; and power supply means which supplies electric power for heat generation to the part of the puncturing part which is formed of the conductor so that heat is generated at the puncturing part for cauterizing the vein around the puncturing part with the puncturing part allowed to puncture the vein in which the varicosis is formed or the organic tissue in the vicinity of the vein.

In the above arrangement, when the puncturing part of the puncturing member is allowed to puncture the vein in which the varicosis is formed and power supply by the power supply means starts, the heat is generated at the puncturing part to cauterize the vein around the puncturing part, thereby occluding the vein. As well, when the puncturing part of the puncturing member is allowed to puncture the organic tissue in the vicinity of the vein in which the varicosis is formed and power supply by the power supply means starts, the heat generated at the puncturing part cauterizes the organic tissue in the vicinity of the vein and also the vein in which the varicosis is formed, thereby occluding the vein. As a result, the bloodstream in the vein in which the varicosis is formed is blocked to cause degeneration of the vein, so that the vein fades away from the surface of the skin. Thus, the varicosis treatment without injecting a sclerosing agent is realized in contrast to the conventional one, with a result that the varicosis treatment causes less or no inflammation over a wide range of the vein and the tissue therearound.

Referring to a second invention, in the varicosis treatment apparatus of the first invention, the puncturing member is formed of a conductor in the form of a needle, the puncturing part is provided at the tip end of the puncturing member, and the power supply means is connected to the base end of the puncturing member.

In the above arrangement, the puncturing member is formed of a conductor, so that the electric power can be supplied to the puncturing part only by connecting the power supply means to the base end of the puncturing member, simplifying the structure of the puncturing member. Further, the puncturing member is in the form of a needle to alleviate the pain at puncture of the puncturing part into the skin and to suppress bleeding. In addition, the cauterized range is minimized, which means cauterization at only necessary part of the vein or the organic tissue in the vicinity thereof.

Referring to a third invention, in the second invention, a part of the puncturing member which is located on the base end side from the puncturing part is covered with an insulator for insulating skin from the puncturing member.

In the above arrangement, the skin is insulated from the puncturing member, so that no electric power is supplied from the power supply means to the skin, thereby preventing cauterization of the skin.

Referring to a fourth invention, the varicosis treatment apparatus of the second invention further includes: a cylinder member having one end from which the puncturing part of the puncturing member is inserted with the other end thereof puncturing skin.

In the above arrangement, a part of the puncturing member which is located on the base end side from the puncturing part is covered with the cylinder member to be out of contact with the skin. Accordingly, even if the heat generated at cauterization of the vein or the organic tissue in the vicinity thereof is transferred to the part of the puncturing member which is located on the base end side from the puncturing part, the heat is suppressed from being transferred to the skin.

Referring to a fifth invention, the varicosis treatment apparatus of the fourth invention further includes: suction means connected to the one end of the cylinder member.

With the above arrangement, connection of the one end of the cylinder member to the suction means enables suction of the blood in the vein after puncture of the other end of the cylinder member into the vein in which the varicosis. This enables cauterization with the vein flattened.

Referring to a sixth invention, in the varicosis treatment apparatus of any of the first to fifth inventions, an output value of the power supply means is set within a range between three watts and eight watts, both inclusive.

In the above arrangement, 3-watt or larger electric power is supplied at a part of the puncturing part which is formed of a conductor. Accordingly, electric power required for cauterizing the vein completely can be obtained at the puncturing part. Further, the thus obtained electric power is eight watts or lower, preventing the organic tissue around the vein from being cauterized undesirably.

Referring to a seventh invention, in the varicosis treatment apparatus of any of the first to sixth inventions, the power supply means includes a switch for staring power to be supplied to the part of the puncturing part which is formed of the conductor and a control circuit for allowing the power to be supplied for two seconds or shorter from operation of the switch and then stopping the supply.

In the above arrangement, operation of the switch stops power supply after supplying the power for two seconds or shorter to a part of the puncturing part which is formed of the conductor. This means no long-time continuation of power supply, thereby preventing undesirable cauterization of the organic tissue around the vein.

### EFFECTS OF THE INVENTION

In the first invention, the puncturing part of the puncturing member is allowed to puncture the vein in which the varicosis is formed or the organic tissue in the vicinity thereof, and the electric power is supplied from the power supply means to the part of the puncturing part which is formed of the conductor for cauterizing the vein around the puncturing part. Accordingly, the varicosis treatment can be performed without using any conventional sclerosing agents. This obviates inflammation over a wide range of the vein and the organic tissue in the vicinity thereof, leading to cosmetically excellent treatment with skin injury prevented from being caused and with pain suppressed.

According to the second invention, with the puncturing member formed of the conductor in the form of a needle, cost reduction of the treatment apparatus is achieved. Further, with the needle-like puncturing member, pain and bleeding can be suppressed, and the cauterized range is minimized, which means in that only a to-be-treated part can be cauterized. Hence, low invasive treatment is achieved.

In the third invention, the insulator insulates the skin from the puncturing member, thereby preventing cauterization of an undesirable part of the skin to achieve low invasive treatment.

In the fourth invention, the puncturing part of the puncturing member is inserted into the cylinder member puncturing the skin, and therefore, the skin is prevented from being damaged by the heat at cauterization of the vein or the organic tissue in the vicinity thereof.

In the fifth invention, which includes the suction means connected to the one end of the cylinder member, the blood in the vein is sucked, thereby flattening the vein to allow the puncturing part to adhere to the inner face of the vein. This increases the efficiency of vein cauterization to reduce a time period required for occluding the vein and to occlude the vein securely, thereby blocking the bloodstream.

In the sixth invention, the electric power in the range between three watts and eight watts, both inclusive, is supplied to the part of the puncturing part which is formed of the conductor, thereby occluding the vein securely and preventing an undesirable part of the organic tissue around the vein from being cauterized.

In the seventh invention, the electric power is supplied to the puncturing part for two second or shorter by operating the switch of the power supply means, thereby reliably preventing an undesirable part of the organic tissue around the vein from being cauterized.

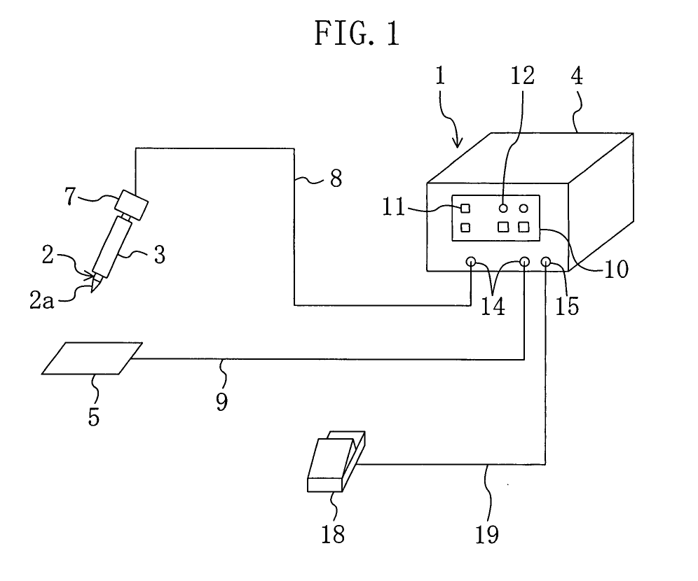

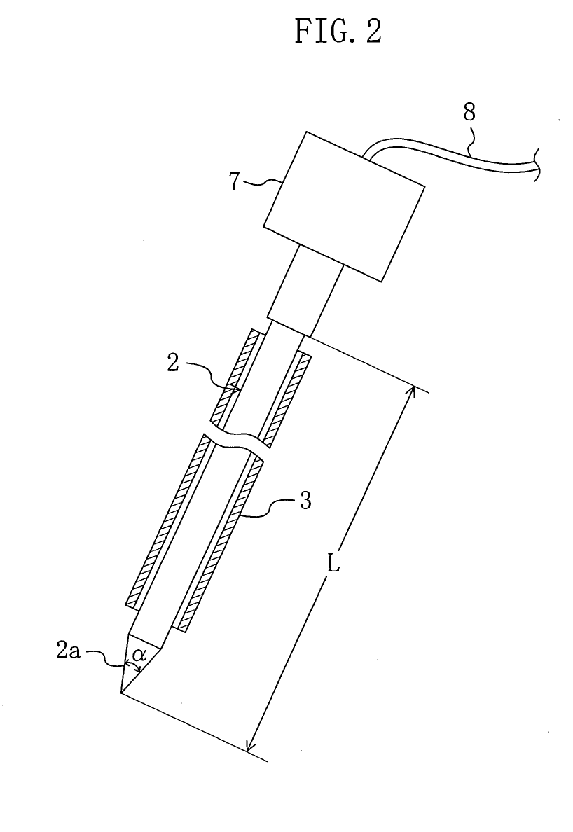

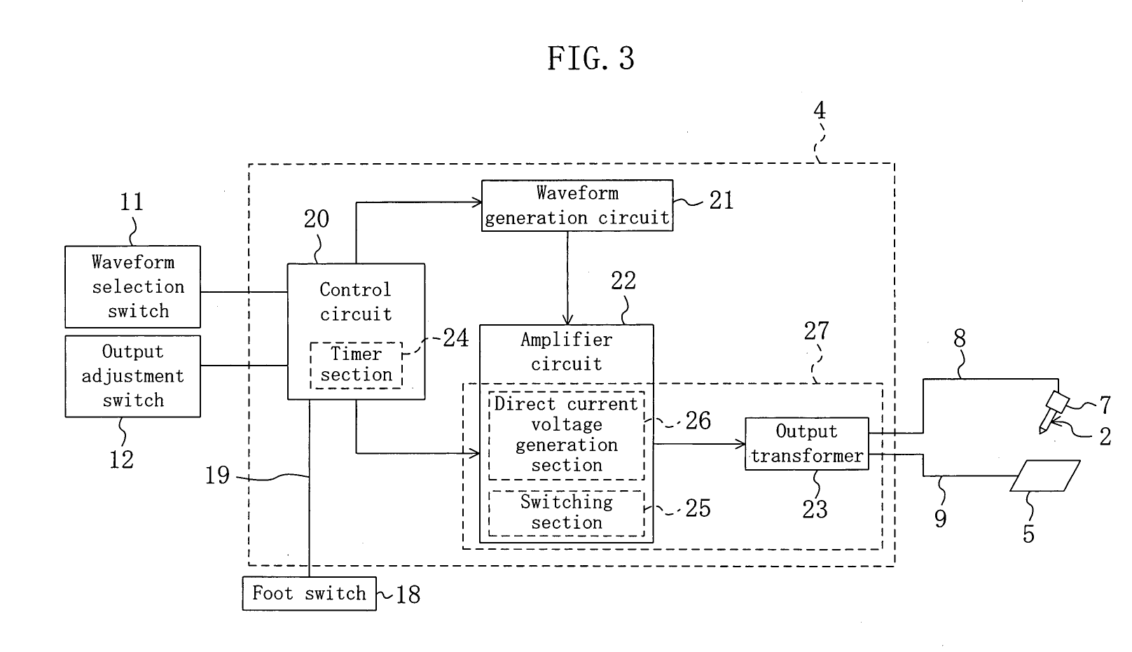

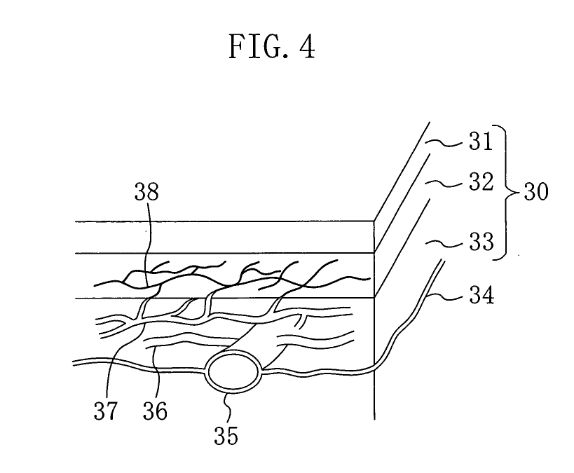

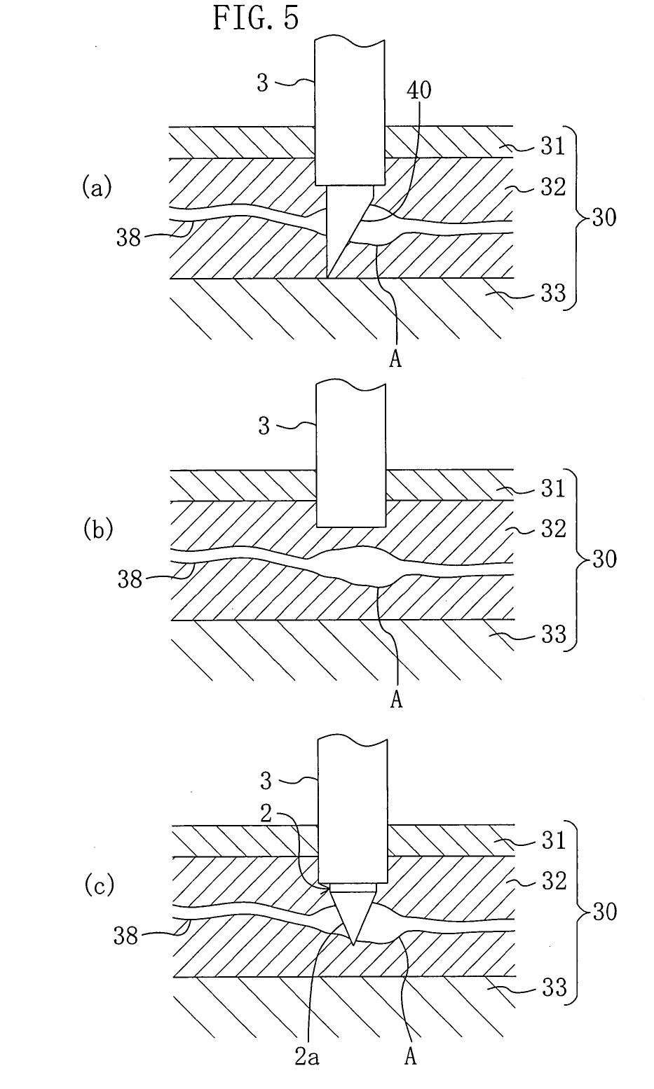

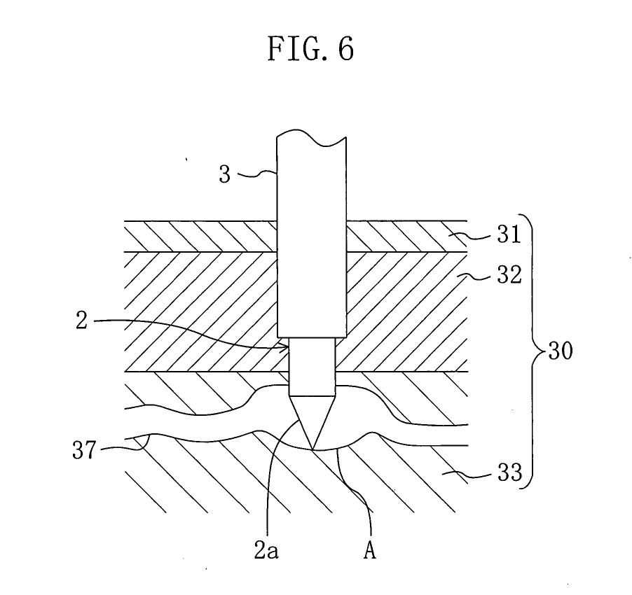

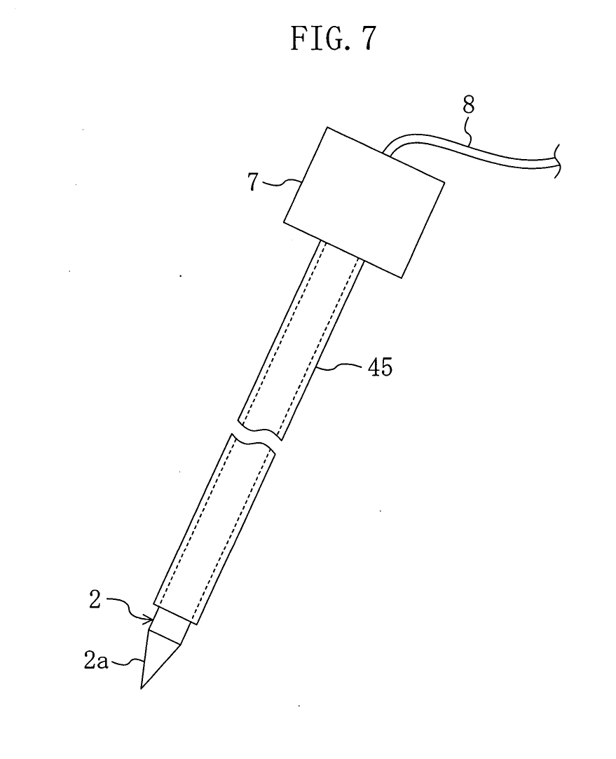

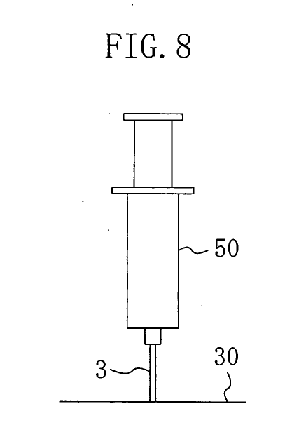

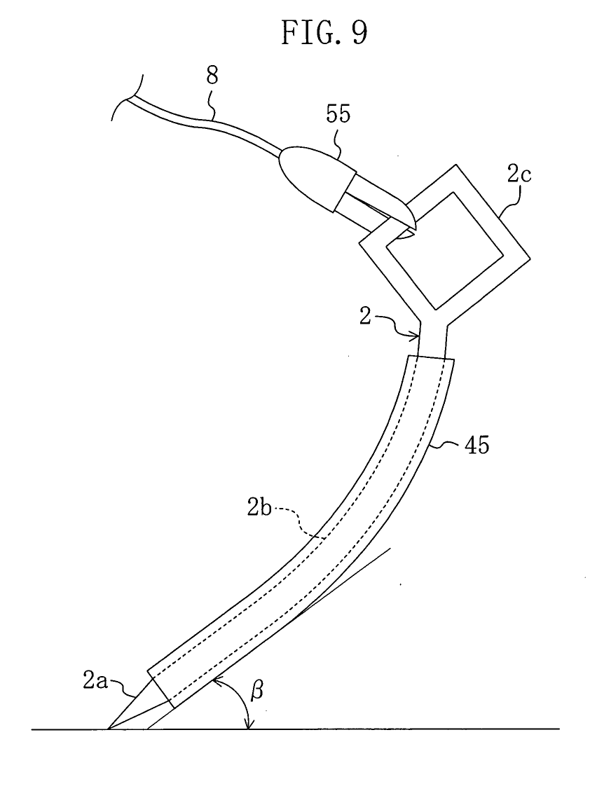

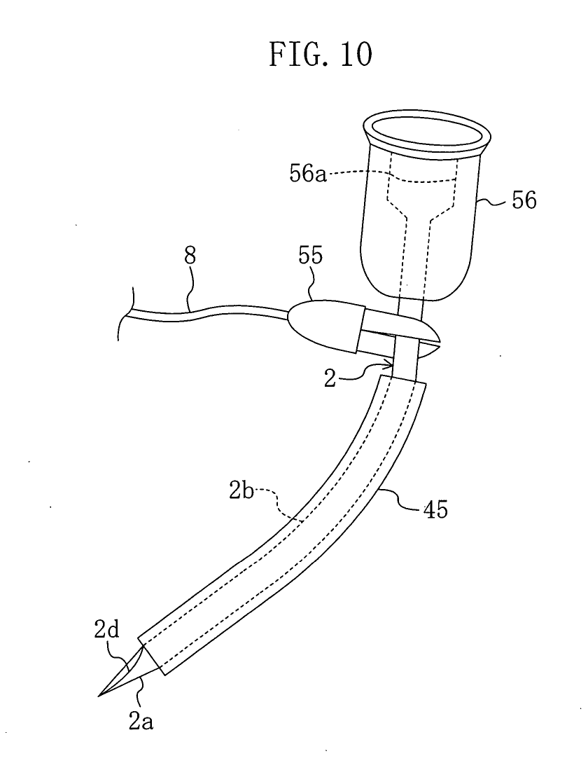

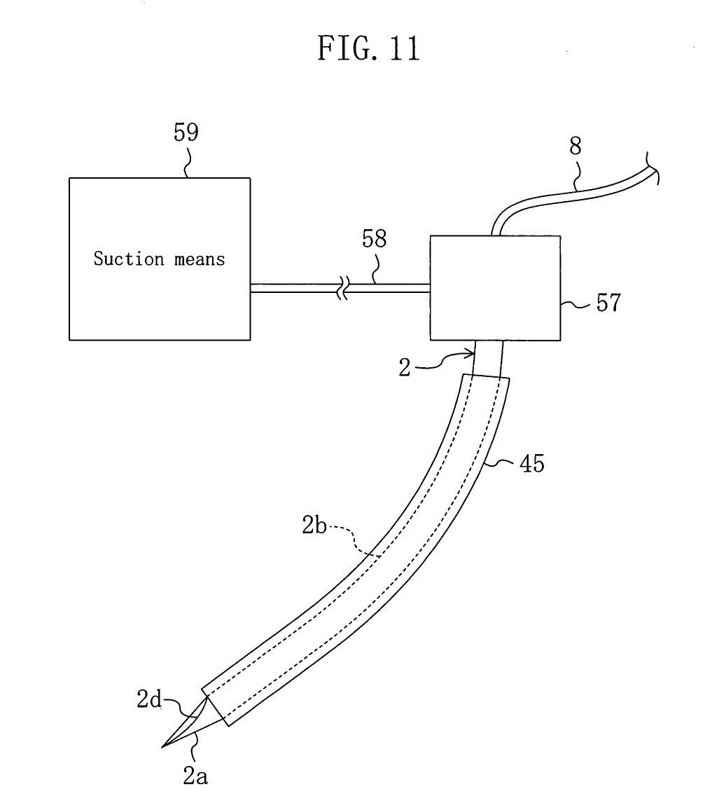

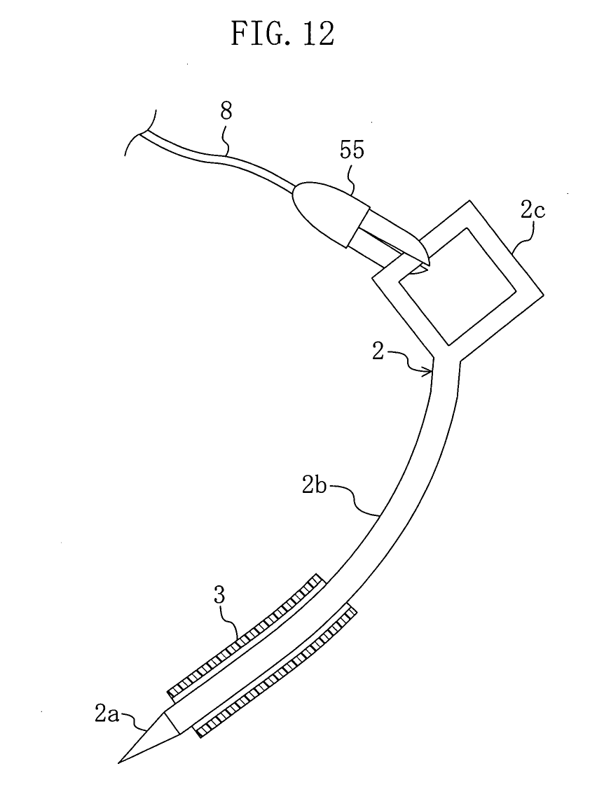

### BRIEF DESCRIPTION OF THE DRAWINGS

[FIG. 1] FIG. 1 is a schematic illustration showing a construction of a varicosis treatment apparatus according to the present invention.

[FIG. 2] FIG. 2 is a partial sectional view showing a puncturing member and a cylinder member in an enlarged scale.

[FIG. 3] FIG. 3 is a block diagram of the varicosis treatment apparatus.

[FIG. 4] FIG. 4 is an anatomical view of skin.

[FIG. 5] FIG. 5 presents enlarged sectional views of skin for showing a procedure for treating a varicosis formed in a vein distributed in dermis, wherein FIG. 5(a) shows a state in which the cylinder member is allowed to puncture the skin together with a needle, FIG. 5(b) shows a state in which the needle is pull out, and FIG. 5(c) shows a state in which a puncturing part of the puncturing member puncture the vein.

[FIG. 6] FIG. 6 is an illustration corresponding to FIG. 5(c) for showing a procedure for treating a varicosis formed in a vein distributed in hypodermal issue.

[FIG. 7] FIG. 7 is a side view of a puncturing member according to Modified Example 1 of the embodiment.

[FIG. 8] FIG. 8 is an explanatory drawing in the case where the blood in a varicosis in a vein is sucked.

[FIG. 9] FIG. 9 is a side view of a puncturing member according to Modified Example 2 of the embodiment.

[FIG. 10] FIG. 10 is a side view of a puncturing member according to Modified Example 3 of the embodiment.

[FIG. 11] FIG. 11 is a side view of a puncturing member according to Modified Example 4 of the embodiment.

[FIG. 12] FIG. 12 is a side view of a puncturing member according to Modified Example 5 of the embodiment.

### EXPLANATION OF REFERENCE NUMERALS

1 treatment apparatus
2 puncturing member
2a puncturing part
3 cylinder member
18 foot switch
20 control circuit
30 skin
45 insulator
50 injection syringe (suction means)
A varicosis

### BEST MODE FOR CARRYING OUT THE INVENTION

An embodiment of the present invention will be described below in detail with reference to the accompanying drawings.

FIG. 1 shows a varicosis treatment apparatus 1 according to the embodiment of the present invention. The varicosis treatment apparatus 1 includes: a puncturing member 2 for puncturing a vein in which a varicosis is formed or organic tissue in the vicinity of the vein; a cylinder member 3 into which the puncturing member 2 is to be inserted; an apparatus main body 4 for supplying electric power to the puncturing member 2; and a counter electrode plate 5 which is connected to the apparatus main body 4 and is to be allowed to be in contact with the surface of an organism.

The puncturing member 2 is made of a metal material as a conductor in the form of a needle, as shown in FIG. 2, and the tip end thereof is tapered and serves as a puncturing part 2a to be allowed to puncture a vein. The puncturing member 2 has a length L in the range between 40 mm and 70 mm, both inclusive, and is arranged so as to be easily handled by the operator. The tapered angle a of the puncturing part 2a when viewed from a side is set within the range between 25° and 35°, both inclusive, so as to be easily puncture the skin.

A gripping portion 7 to be gripped by the operator is provided at the base end of the puncturing member 2 so that the operator can smoothly manipulate the puncturing member 2. One end of a connection cord 8 extending from the apparatus main body 4 is fixed to the gripping portion 7 so that the one end of the connection cord 8 allows the base end of the puncturing member 2 to be conductive. The counter electrode plate 5 is formed of a conductor to be conductive through one end of another connection cord 9.

The cylinder member 3 is a generally-called metal-made indwelling needle and has an inner diameter larger than the outer diameter of the puncturing member 2 and a length shorter than the puncturing member 2. Each end face in the longitudinal direction of the cylinder member 3 extends in the direction intersecting at a right angle with the axial line of the cylinder member 3.

Further, as shown in FIG. 1, there are provided at an operation panel 10 of the apparatus main body 4 a waveform selection switch 11 for selecting a waveform of output electric current between a continuous rectangular waveform and a discontinuous rectangular waveform, an output adjustment switch 12 for adjusting magnitude of the output electric current, and the like. In addition, the operation panel 10 includes terminals 14 into which the other end of the connection cord 8 for the puncturing member 2 and the other end of the connection cord 9 for the counter electrode plate 5 are plugged and another terminal 15 into which a connection cord 19 for a foot switch 18 that starts power supply to the puncturing member 2 is plugged. Power supply means of the present invention is composed of the apparatus main body 4, the counter electrode plate 5, the connection cords 8, 9, 19, and the foot switch 18.

Inside the apparatus main body 4, as shown in FIG. 3, there are provided: a control circuit 20; a waveform generation circuit 21 controlled by the control circuit 20 for generating a waveform of the output electric current; a variable amplifier circuit 22 for changing a waveform signal from the waveform generation circuit 21 to an output waveform; and an output transformer 23 for boosting a signal output from the variable amplifier circuit 22

The control circuit 20 are connected to the waveform selection switch 11, the output adjustment switch 12, and the foot switch 18 so as to detect each operation of the switches 11, 12, 18. Upon detection of the operation of the foot switch 18, the control circuit 20 sends to the waveform generation circuit 21 a waveform selection signal as a drive parameter corresponding to a waveform selected by the waveform selection switch 11. The waveform generation circuit 21 that has received the waveform selection signal generates a waveform signal corresponding to the waveform selection signal. The thus generated waveform signal is input to a switching section 25 that the variable amplifier circuit 22 includes, thereby activating the switching section 25.

Further, the control circuit 20 includes a timer section 24 that starts its operation at the time point when the foot switch 18 is operated. While detecting, with the use of the timer section 24, a lapse of a predetermined period from the time point when the foot switch 18 is operated, the control circuit 20 sends the waveform selection signal until the predetermined time elapses from the time point when the foot switch 18 is operated, and then, stops sending of the waveform selection signal. In other words, when the predetermined period lapses from the time point when the foot switch 18 is operated, the waveform generation circuit 21 stops sending the waveform signal so that the power supply from the apparatus main body 4 is stopped. The predetermined period set in the timer section 24 can be changed by, for example, a switch or the like provided at the operation panel 10 arbitrarily in the range of two seconds or shorter, wherein it is set to one second in the present embodiment.

Moreover, the control circuit 20 sends to a direct current voltage generation section 26 of the variable amplifier circuit 22 a voltage signal as a drive parameter corresponding to an output value set by the output adjustment switch 12. The direct current voltage generation section 26 generates direct current voltage corresponding to the input voltage signal. The direct current voltage generated at the direct current voltage generation section 26 is applied to a primary winding (not shown) of the output transformer 23.

The direct current voltage generation section 26, the output transformer 23, and the switching section 25 compose an inverter circuit 27. In the inverter circuit 27, when a signal from the switching section 25 and the direct current voltage from the direct current voltage generation section 26 are input to the output transformer 23, switching operation for changing the electric current flowing in the output transformer 23 at high speed is performed to convert the electric current to high-frequency electric power. The frequency of the electric power obtained at conversion is in the range between 300 kHz and 10 MHz, and accordingly, no ventricular fibrillation of a hart is caused even if the electric power obtained at the inverter circuit 27 flows into the human body. The output adjustment switch 12 can set the high frequency electric power obtained at the inverter circuit 27 arbitrarily within the range between three watts and eight watts, both inclusive, wherein it is set to five watts in the present embodiment.

The voltage applied to the primary winding of the output transformer 23 is boosted and then is applied to a secondary winding. The secondary winding is connected to the connection cord 8 for the puncturing member 2 and the connection cord 9 for the counter electrode plate 5. Accordingly, when the puncturing part 2a of the puncturing member 2 is allowed to puncture the vein or the organic tissue in the vicinity of the vein and the foot switch 18 is operated with the counter electrode plate 5 allowed to be in contact with the surface of the patient's body, a secondary side circuit of the output transformer 23 is in a closed loop state, so that the high frequency electric power generated at the apparatus main body 4 flows in the loop. Since the control circuit 20 with the timer section 24 composes means for automatically stopping the power supply to the puncturing part 2a, only one-time operation of the foot switch 18 causes the power to be supplied for one second and then to be stopped automatically.

It is noted that a switch other than the foot switch 18 may be provided for starting power supply by the switch.

Description will be given next to a procedure for treatment of a varicosis formed in a vein distributed in human skin with the use of the varicosis treatment apparatus 1 constructed as above. Before describing the treatment procedure, veins distributed in human skin will be described with reference to FIG. 4. The skin 30 is composed of epidermis 31, dermis 32, and hypodermal tissue 33, wherein the hypodermal tissue 33 includes a fascia 32 extending along the dermis 32. In the hypodermal tissue 33, there are distributed a saphenous vein 35 integral with the fascia 34, a segmental vein 36 branching from the saphenous vein 35, and a vein 37 located on the dermis 32 side of the segmental vein 36 and formed of a small vessel. In the dermis 32, a vein 38 formed of a vessel thinner than the vein 37 of the hypodermal tissue 33 is distributed.

In the case where a reticular type varicosis A is formed in the vein 38 distributed in the dermis 32, the cylinder member 3 is allowed to puncture first the epidermis 31 and up to a part of the dermis 32 on the epidermis 31 side, as shown in FIG. 5(a). In this puncture, when a needle 40 is inserted into the cylinder member 3 and the cylinder member 3 is allowed to puncture the epidermis 31 and the dermis 32 with the tip end of the needle 40 protruding from the cylinder member 3, the tip end of the needle 40 dissects the epidermis 31 and the dermis 32 so that the end part of the cylinder member 3 can puncture the epidermis 31 and the dermis 32 effortlessly. The end part of the cylinder member 3 is allowed to puncture the dermis 32 until it reaches a point in the vicinity of the varicosis A in the dermis 32, and then, the needle 40 is pulled out from the cylinder member 3, as shown in FIG. 5(b).

Subsequently, as shown in FIG. 5(c), the puncturing member 2 is inserted into the cylinder member 3 to allow the puncturing part 2a of the puncturing member 2 to puncture the vein 38 in which the varicosis A is formed. Since the end part of the cylinder member 3 is positioned in the vicinity of the varicosis A, the puncturing part 2a is guided to the vicinity of the varicosis A only by inserting the puncturing member 2 into the cylinder member 3. Before or after puncturing the vein 38 by the puncturing member 2, the counter electrode plate 5 is allowed to be in contact with the surface of the patient's body.

Thereafter, the waveform selection switch 11 and the output adjustment switch 12 of the apparatus main body 4 are operated to select a desired waveform and set a desired output, respectively. When the foot switch 8 is operated then, the electric power is supplied for only one second to the puncturing member 2 from the apparatus main body 4. The electric current flowing during the period generates heat at the puncturing part 2a, so that vein 38 around the puncturing part 2a is cauterized by the heat generated at the puncturing part 2a. Cauterization of the vein 38 causes the inner wall of the vein 38 to adhere and to be occluded. If the cauterization is insufficient, the foot switch 18 is operated again to supply the electric power only for one second for cauterizing the vein 38. The above operation is repeated until the inner wall of the vein 38 adheres completely. In this way, the vein 38 in which the bloodstream is blocked is degenerated and then disappears in due course.

Referring to the aforementioned reticular type varicosis A, multiple veins 38 in which varicoses are formed are located close to each other. Therefore, in order to treat the reticular type varicosis A, after one of the veins 38 is cauterized as above, the other veins 38 are cauterized as well one by one. With the puncturing member 2 having an outer diameter smaller than the inner diameter of the cylinder member 3, the other veins 38 can be cauterized only by moving the puncturing part 2 within the skin 30 with the cylinder member 3 remaining puncturing the skin 30.

In order to treat a varicosis A formed in the vein 37 distributed in the hypodermal tissue 33 deeper than the dermis 32, the cylinder member 3 is allowed to puncture a part deep in the dermis 32, and then, the puncturing member 2 is inserted into the cylinder member 3 so as to allow the puncturing part 2a to puncture the vein 37 for cauterizing the vein 37 as in the aforementioned manner.

Thus, according to the varicosis treatment apparatus 1 of the present embodiment, the varicosis A is treated by cauterizing the vein 37 or 38 without using the conventional sclerosing agent. This prevents inflammation of the vein 37 or 38 and the organic tissue around the vein 37 or 38 from spreading over a wide range. Accordingly, cosmetically excellent treatment can be achieved with skin injury prevented and with pain suppressed.

With the puncturing member 2 formed of a conductor in the form of a needle, only connection of the base end of the puncturing member 2 to the apparatus main body 4 attains power supply to the puncturing part 2a. This lower the cost of the puncturing member 2 to lead to cost reduction of the treatment apparatus 1. Further, with the needle-like puncturing part 2a, pain and bleeding, which would have been accompanied by the treatment, can be suppressed, and the cauterized range is minimized with only a to-be-cauterized part cauterized. Hence, low invasive treatment can be achieved.

Moreover, the vein 37 or 38 is cauterized under a state that the puncturing member 2 is inserted into the cylinder member 3 puncturing in the skin 30 so that only the puncturing part 2a protrudes from the end of the cylinder member 3 with a part of the puncturing member 2 which is located on the base end side from the puncturing part 2a covered with the cylinder member 3. Accordingly, even if the heat generated at cauterization is transferred to a part of the puncturing member 2 which is located on the base end side from the puncturing part 2a, the heat is prevented from being transferred directly to the epidermis 31 and the organic tissue therearound. Thus, the epidermis 31 and the tissue therearound can be prevented from damaged by the heat at cauterization.

Since the electric power output from the apparatus main body 4 is set at three watts or higher, electric power sufficient for cauterizing the vein 37 or 38 can be supplied to the puncturing member 2, resulting in reliable occlusion of the vein 37 or 38. Further, with the electric power output from the apparatus main body 4 is set at eight watts or lower, undesirable cauterization of the organic tissue around the vein 37 or 38 is prevented, leading to low invasive treatment.

One-time operation of the foot switch 18 causes the electric power to be supplied for only one second to the puncturing part 2a, which means no continuous power supply for a long period of time. Hence, undesirable cauterization of the organic tissue around the vein 37 or 38 is prevented, leading to low invasive treatment as well. Two seconds or shorter time period for power supply through the operation of the foot switch 18 achieves equivalent effects.

It is noted that though the puncturing part 2a is allowed to puncture the vein 37 or 38 for directly cauterizing the vein 37 or 38 in the above embodiment, the vein 37 or 38 may be cauterized indirectly by allowing the puncturing part 2a to puncture the organic tissue in the vicinity of the vein 37 or 38, that is, the skin 30 for cauterizing the vein 37 or 38. Heat used for cauterizing the organic tissue in the vicinity of the vein 37 or 38 is transferred to the vein 37 or 38 to cauterize and occlude the vein 37 or 38.

Further, the cylinder member 3 may be made of a material having an insulation characteristic, such as resin or the like. This prevents reliably the electric current flowing in the puncturing member 2 from flowing into the skin 30. In the case where the cylinder member 2 is made of resin, the heat at the puncturing part 2a of the puncturing member 2 is prevented reliably from being transferred to the skin 30 when compared with the cylinder member made of metal.

Furthermore, though the above embodiment uses the cylinder member 3, the treatment for the varicosis A can be performed without using the cylinder member 3, as in Modified Example shown in FIG. 7. In this case, a part of the puncturing member 2 which is located on the base side from the puncturing part 2a is covered with an insulator 45. The insulator 45 insulates the skin 30 from a part of the puncturing member 2 which is other than the puncturing part 2a to prevent the electric current from the apparatus main body 1 to flow into the skin 30, preventing the skin 30 from being cauterized. Thus, lower invasive treatment can be achieved.

Optionally, the blood in the vein 37 or 38 may be sucked before cauterization of the vein 37 or 38. To do so, an injection syringe 50 as suction means is attached to the end on opposite side of the cylinder member 3 from the puncturing part 2a, as shown in FIG. 8. The injection syringe 50 is also a component of the treatment apparatus 1. Manipulation of the injection syringe 50 in a sucking direction with the cylinder member 3 puncturing the vein 37 or 38 causes the blood in the vein 37 or 38 to be sucked into the cylinder of the injection syringe 50. This flattens the vein 37 or 38 and less or no blood remains in the vein 37 or 38, allowing the puncturing part 2a to adhere to the inner face of the vein 37 or 38. Accordingly, the puncturing part 2a cauterizes effectively the vein 37 or 38, and the vein 37 or 38 is occluded surely within a shortened period of time for cauterizing the vein 37 or 38. Hence, further lower invasive treatment can be achieved. The suction means in this case is not limited to the injection syringe 50 and any sucking apparatus such as an blood aspirator used in general medical worksites may be used.

Further optionally, a curved portion 2b may be formed in the middle in the longitudinal direction of the puncturing member 2, as in Modified Example 2 shown in FIG. 9. The curved portion 2b is so formed that the inclination angle β at the tip end part of the puncturing member 2 with respect to the horizontal plane is in the range between 30° and 45°, both inclusive, in the state where the base end part of the puncturing member 2 is set substantially perpendicular. The puncturing member 2 is obtained by processing a solid bar having a diameter substantially the same as a medical injection syringe 30 G (gate) to 26 G and a total length of approximately 60 mm. Further, the curved portion 2b is so formed that the puncturing member 2 has a length in the vertical direction in the range between 40 mm and 50 mm, both inclusive, in the state where the base end part of the puncturing member 2 is set substantially perpendicularly.

A part of the puncturing member 2 which is located on the base end side from the puncturing part 2a is covered with the insulator 45 made of resin, similarly to that in Modified Example 1, with the base end part of the puncturing member 2 exposed. A rectangular frame 2c is provided at the base end of the puncturing member 2, wherein the frame 2c is exposed from the insulator 45 as well. Further, in Modified Example 2, an alligator clip 55 is provided at the tip end of the connection cord 8 extending from the operation panel 10 of the apparatus main body 4. When the alligator clip 55 clips the frame 2c of the puncturing member 2, the connection cord 8 is connected electrically to the puncturing member 2. The alligator clip 55 for connecting the puncturing member 2 to the connection cord 8 facilitates attachment and detachment of the connection cord 8 to and from the puncturing member 2. This facilitates an operation for cleaning and disinfection the puncturing member 2 detached from the connection cord 8 after treatment and an operation for exchanging puncturing members 2 according to the symptom of the varicosis A.

In Modified Example 2, the varicosis A is occluded by cauterizing the organic tissue in the vicinity of the varicosis A. Specifically, Modified Example 2 is applied to treatment of a web type varicosis formed in the thin vein 38 distributed in the upper part of the skin 30. The varicosis A of the web type ranges wide in a direction along the surface of the skin 30 and the vein 38 is thin, which involves difficulty in puncturing the vein 38 by the puncturing part 2a. In order to tackle this difficulty, the apparatus 1 of Modified Example 2 is used in such a manner that the puncturing member 2 is allowed to puncture the skin 30; when the puncturing part 2a reaches the vicinity of the varicosis A, the vein 38 is cauterized by the heat used for cauterizing the skin 30 as the organic tissue; and then the puncturing part 2a is pushed in a direction that the varicosis A spreads, that is, the direction along the surface of the skin 30 for cauterizing the other part of the vein 38. Further, when the puncturing member 2 is moved along the surface of the skin 30 so that the direction of the puncturing part,2a agrees with the longitudinal direction of the vein 38, the heat generated at the puncturing part 2a can be transferred to a comparatively wide range of the vein 38 to cauterize the wide range of the vein 38.

For pushing the puncturing part 2a along the surface of the skin 30 as described above, it is necessary to set the tip end part of the puncturing member 2 in the direction along the surface of the skin 30. When the tip end part of the puncturing member 2 is set in the direction along the surface of the skin 30, the base end part of the puncturing member 2, which includes the curved portion 2b, sticks out from the surface of the skin 30 almost perpendicularly to the skin 30 and is separated from the surface of the skin 30. Accordingly, the operator can hold the base end part of the puncturing member 2 easily, facilitating manipulation of the puncturing member 2, as described above, for cauterizing a wide range of the vein 38. Further, when the operator holds the base end part of the puncturing member 2 so that the tip end thereof is set substantially perpendicular to the skin 30, resistance in puncturing the skin 30 by the puncturing member 2 is reduced.

Still optionally, as in Modified Example 3 shown in FIG. 10, the puncturing member 2 may be so hollowed that both ends thereof in the longitudinal direction are opened. Similarly to that of Modified Example 2, the puncturing member 2 includes the curved portion 2b and is covered with the insulator 45 so that the base end part of the puncturing member 2 which is not covered with the insulator 45 is clipped by the alligator clip 55. The puncturing part 2 is obtained by processing a hollowed bar having a diameter substantially the same as a medical injection syringe 22 G or 21 G and a total length of approximately 60 mm. The curved portion 2c is so formed that the puncturing member 2 has a length in the vertical direction in the range between 40 mm and 50 mm, both inclusive, in the state where the base end part of the puncturing member 2 is set substantially perpendicularly.

In Modified Example 3, the outer face of the puncturing part 2a forms an opening at the tip end of the puncturing member 2 and serves as a suction port 2d for sucking the blood in the vein 37 or 38. On the other hand, at the base end of the puncturing member 2, a joint member 56 is mounted for joining an injection syringe (not shown). The joint member 56 is in the form of a cylinder as a whole, the inside of which communicates with the other opening at the base end of the puncturing member 2. A tapered face 56a with an inclination of approximately 6/100 is formed at the inner face of the joint member 56 so that the tip end part of the injection syringe is fitted therein and joined to the puncturing member 2. In the state where the injection syringe is joined to the puncturing member 2, the inside of the puncturing member 2 and the inside of the injection syringe communicate with each other.

In Modified Example 3, the vein 37 or 38 in which the varicosis A is formed is cauterized to be occluded. In detail, the blood in the varicosis A is sucked through the suction port 2d by manipulating the injection syringe after puncturing the varicosis A by the puncturing part 2a of the puncturing member 2, and then, the vein 37 or 38 is cauterized directly. In direct cauterization of the vein 37 or 38, the puncturing part 2a can adhere to the inner face of the vein 37 or 38 with less or no blood remaining in the varicosis A, thereby achieving effective cauterization of the vein 37 or 38 by the puncturing part 2a. As a result, the vein 37 or 38 is occluded surely within a shortened time period for cauterizing the vein 37 or 38, achieving further lower invasive treatment. The joint member 56 may be joined to any suction means such as a blood aspirator used in general medical worksites rather than the injection syringe.

In Modified Example 3, similarly to Modified Example 2, the operator can hold easily the base end part of the puncturing member 2 for pushing the puncturing part 2a of the puncturing member 2 in the direction that the varicosis A spreads, facilitating manipulation of the puncturing member 2. Further, when the tip end of the puncturing member 2 is set to be substantially perpendicular to the skin for allowing the puncturing member 2 to puncture the skin 30, resistance in puncture can be reduced.

Alternatively, a connection block 57 for connecting the suction means 59 and the connection cord 8 to the base end of the puncturing member 2 may be provided as in Modified Example 4 shown in FIG. 11. The puncturing member 2 of Modified Example 4 is structured just the same as the puncturing member 2 of Modified Example 3. The connection block 57 is provided with a connector (not shown) conductive to the base end of the puncturing member 2, and the connection cord 8 is connected to the connector. Further, a suction hose 58 is mounted at the connection block 57 for communicating with the opening at the base end of the puncturing member 2 so that the inside of the puncturing member 2 and the suction means 59 communicate with each other through the suction hose 58. In Modified Example 4, similarly to Modified Example 3, the vein 37 or 38 is cauterized after the blood in the vein 37 or 38 in which the varicosis A is formed is sucked.

Still alternatively, as in Modified Example 5 shown in FIG. 12, a cylinder member 3 may be provided into which the puncturing member 2 including the curved portion 2b is to be inserted. When the cylinder member 3 is made of resin, such as Teflon (trademark), or the like, for example, the electric current flowing in the puncturing member 2 is prevented from flowing into the skin 30 and the heat at the puncturing part 2a of the puncturing member 2 is prevented reliably from being transferred to the skin 30.

In addition, with the use of the treatment apparatus 1, any varicosis formed in the saphenous vein 35 or the segmental vein 36 in the skin 30 can be treated.

### INDUSTRIAL APPLICABILITY

As described above, the varicosis treatment apparatus according to the present invention can be used for treatment of a varicosis formed in a vein distributed in organic skin, for example.

1. A varicosis treatment apparatus comprising:
a puncturing member including a puncturing part for puncturing a vein in which a varicosis is formed or an organic tissue in the vicinity of the vein, at least a part of the puncturing part being formed of a conductor; and
power supply means which supplies electric power for heat generation to the part of the puncturing part which is formed of the conductor so that heat is generated at the puncturing part for cauterizing the vein around the puncturing part with the puncturing part allowed to puncture the vein in which the varicosis is formed or the organic tissue in the vicinity of the vein.

2. The varicosis treatment apparatus of claim 1,
wherein the puncturing member is formed of a conductor in the form of a needle, the puncturing part is provided at the tip end of the puncturing member, and the power supply means is connected to the base end of the puncturing member.

3. The varicosis treatment apparatus of claim 2,
wherein a part of the puncturing member which is located on the base end side from the puncturing part is covered with an insulator for insulating skin from the puncturing member.

4. The varicosis treatment apparatus of claim 2, further comprising:
a cylinder member having one end from which the puncturing part of the puncturing member is inserted with the other end thereof puncturing skin.

5. The varicosis treatment apparatus of claim 4, further comprising:
suction means connected to the one end of the cylinder member.

6. The varicosis treatment apparatus of claim 1,
wherein an output value of the power supply means is set within a range between three watts and eight watts, both inclusive.

7. The varicosis treatment apparatus of claim 1,
wherein the power supply means includes a switch for staring power to be supplied to the part of the puncturing part which is formed of the conductor and a control circuit for allowing the power to be supplied for two seconds or shorter from operation of the switch and then stopping the supply.
